(19) United States
(12) Patent Application Publication
(10) Pub. No.: US 2020/0308569 A1
(43) Pub. Date: Oct. 1, 2020

(54) METHODS AND APPARATUS FOR PURIFYING RNA

(71) Applicant: MODERNATX, INC., Cambridge, MA (US)

(72) Inventor: Benjamin Frank GELDHOF, Winthrop, MA (US)

(73) Assignee: ModernaTX, Inc., Cambridge, MA (US)

(21) Appl. No.: 16/311,442

(22) PCT Filed: Jun. 21, 2017
(86) PCT No.: PCT/US2017/038534

§ 371 Date: Dec. 19, 2018

Related U.S. Application Data
(60) Provisional application No. 62/354,376, filed on Jun. 24, 2016.

(51) Int. Cl. C12N 15/10; B01L 3/00; B01L 7/00; G01N 1/40

(52) U.S. Cl. CPC C12N 15/101; C12N 15/1017; B01L 3/502; B01L 7/00; G01N 1/4077; G01N 1/405; B01L 2300/049; B01L 2400/06; B01L 2400/0487; B01L 2300/1805; B01L 2300/06; G01N 2001/4088; B01L 2300/047

(57) ABSTRACT

The invention features methods and apparatus for purifying a sample including an RNA (e.g., an RNA having a poly(A) sequence, such as an mRNA). Methods of the invention involve adding a liquid to a sample container including the sample and a solid affinity material (e.g., oligo(dT) or hydroxyapatite), agitating the contents of the sample, removing the liquid, and eluting the RNA of interest from the sample container. The apparatus of the invention include a sample container and one or more components such as a reservoir, collection container, pressure generating source, valve assembly, and agitation mechanism. Certain apparatus include a rotatable magnet or magnetizable component. The methods and apparatus described herein are useful in providing RNA (e.g., RNA having a poly(A) sequence, such as mRNA) in high yield and with high purity using less solvent than conventional purification techniques.

### BACKGROUND OF THE INVENTION

Ribonucleic acids (RNA) play important roles in the regulation, coding, and expression of genes. Cellular organisms include many different kinds of RNA, each of which may perform a unique role within a biological system. Certain RNA molecules include a sequence of nucleotides including only adenine nucleobases. So-called poly(A) sequences (also known as poly(A) tails or regions) are thought to stabilize the RNA and to facilitate the termination of transcription, the export of the RNA from cellular regions such as the nucleus, and translation. An RNA having a poly(A) sequence may be a messenger RNA (mRNA). mRNA molecules encode polypeptides and are responsible for gene expression. In recent years, RNA molecules having poly(A) sequences (e.g., mRNA) have been identified as potentially useful therapeutic and/or prophylactic agents for a variety of pharmaceutical applications. However, methods and apparatus for purifying such RNA molecules in high purity and yield are lacking. RNA having poly(A) sequences are often components of samples including substantial amounts of other kinds of RNA in addition to other contaminants. Further, current methods and apparatus for purifying RNA having poly(A) sequences may require large amounts of solvent. Thus, improved methods and apparatus for purifying RNA having poly(A) sequences on large and small scales with higher purity and higher yield and using less solvent are needed.

### SUMMARY OF THE INVENTION

The present invention provides methods and apparatus for purifying a sample including an RNA, e.g., having a poly(A) sequence (e.g., an mRNA).

In one aspect, the invention provides a method of purifying a sample including an RNA, e.g., having a poly(A) sequence by adding a liquid to a sample container comprising the sample and a solid affinity material that binds to the RNA; agitating the contents of the sample container to mix the RNA and the solid affinity material; removing the liquid from the sample container; and eluting the RNA from the solid affinity material.

In some embodiments, the RNA, e.g., having a poly(A) sequence, is an mRNA. An mRNA may include one or more of a 5′ cap structure, a Kozak sequence, a 3′ UTR, and a 5′ UTR, and may include one or more alternative nucleosides or nucleotides.

The solid affinity material may include oligo(dT) (for RNA having a poly(A) sequence) or hydroxyapatite. Particles including oligo(dT) or hydroxyapatite may have a size of about 10-100 μm, e.g., 30-60 μm. For example, the particles may have a size of about 40 or 45 μm.

The sample container may include a substantially cylindrical portion and/or a substantially conical portion, e.g., disposed at the bottom of a cylindrical portion. In some embodiments, the sample container has an opening disposed at or near its top and an opening disposed at or near its bottom. The sample container may have a porous filter or frit disposed at or near its bottom.

Before liquid is introduced to the sample container, the sample may be loaded onto the top of a packed bed of the solid affinity material. Liquid may be introduced into the sample container from the bottom of the container.

In some embodiments, the contents of the sample container are agitated by introducing an inert gas into the container. The inert gas may be air, nitrogen, argon, or carbon dioxide. In other embodiments, the agitation is by sonication. In other embodiments, the sample container includes a mechanical agitator (e.g., a magnetic disk, e.g., a ferromagnetic disk), and agitation includes moving the agitator (e.g., using an electromagnetic field).

The sample container may be disposed above a positive pressure source to prevent leaking of the liquid.

Adding a liquid to the sample container and agitating the contents of the sample container, in combination, may disrupt the packed bed and mix the solid affinity material with the liquid, and removing liquid from the sample container may result in reformation of the packed bed. In some instances, after removing liquid from the sample container, the container is substantially free of the liquid, e.g., less than 5% of the volume of liquid remains.

In some embodiments, the method is repeated at least once prior to elution.

In another aspect, the invention features an RNA, e.g., having a poly(A) sequence, purified using the methods described herein.

In a further aspect, the invention features an apparatus for purifying a sample including an RNA, e.g., having a poly(A) sequence, the apparatus including a sample container for depositing the sample and having a first opening disposed at or near its top and a second opening disposed at or near its bottom; a reservoir for housing a liquid; and a valve assembly, wherein the valve assembly is fluidically connected to the second opening, the reservoir, an outlet for collection or waste, and an inlet for an inert gas, wherein the valve assembly is configured to allow, separately, liquid transfer from the sample container and the reservoir, liquid transfer from the sample container and the outlet, and gas transfer from the inlet to the sample container.

The apparatus may also include a pump or connection for a pressure source. The pump may be, for example, a peristaltic pump or a syringe pump. The apparatus may further include a computer programmed to have components of the apparatus operate in a specific sequence, and/or a heater for the sample container. The sample container may include, or be configured to accept, a solid affinity material such as oligo(dT) or hydroxyapatite.

The apparatus may include one or more additional sample containers, each having a first opening disposed at or near its top and a second opening disposed at or near its bottom, wherein the valve assembly is fluidically connected to the second opening, the reservoir, an outlet for collection or waste, and an inlet for an inert gas, wherein the valve assembly is configured to allow, separately, liquid transfer from the sample container and the reservoir, liquid transfer from the sample container and the outlet, and gas transfer from the inlet to the sample container.

In yet another aspect, the invention features an apparatus for purifying a sample including an RNA, e.g., having a poly(A) sequence, the apparatus including a sample container for depositing the sample and having a first opening disposed at or near its top and a second opening disposed at or near its bottom; a rotatable magnet disposed below the sample container; and a positive pressure source disposed below the sample container.

The apparatus may also include a reservoir for a liquid in fluid communication with the first opening, and/or an array for holding collection containers to capture eluate from the second opening. The apparatus may further include a pressure source configured to cause liquid in the sample container to exit the second opening. The sample container may include, or be configured to accept, a solid affinity material such as oligo(dT) or hydroxyapatite.

### BRIEF DESCRIPTION OF THE DRAWINGS

FIG. 1 is a diagram of a sample container including a first tapered cylindrical portion, a conical portion, and a second tapered cylindrical portion. The sample container includes a porous disk (e.g., a frit) near its bottom that prevents solid materials from exiting the bottom of the sample container.

FIG. 2 is a diagram of an apparatus including a sample container, pump, collection array, air port, valve assembly, and reservoirs for buffers and reagents. The other components of the apparatus are each fluidically connected to the valve assembly, which can be configured to permit controlled transfer of materials between the various components.

FIG. 3 is an illustration of an apparatus including multiple sample containers, collection containers for collecting materials from the sample containers, and a reservoir for a buffer or reagent. Sample containers are in contact with a temperature control element. The collection containers are disposed within an array capable of transferring the collection containers between different locations in the apparatus, e.g., to permit collection in different containers. Tubing connecting various components is not shown.

FIG. 4 shows two versions of an apparatus of the invention. Both versions include sample containers, buffer reservoirs, pumps with valves, and an array for collection containers. The version at left also includes a temperature control element in contact with the sample containers.

FIG. 5 shows an apparatus including multiple sample containers, each containing a disk (e.g., a magnetic disk), disposed above a positive pressure source and an array of collection containers. One or more reservoirs including buffers and reagents are disposed above the sample containers.

FIG. 6 is an electropherogram of a sample purified using the methods of the invention. RNA having a poly(A) sequence is represented as the sharp peak centered at 878 nucleotides. Purity is determined as the area under the curve in a band +/−15% of the main peak.

### DETAILED DESCRIPTION

The invention relates to methods and apparatus for purifying a sample including an RNA, e.g., an RNA having a poly(A) sequence such as an mRNA, and RNA purified using the same. The methods described herein include adding a liquid to a sample container including the sample and a solid affinity material (e.g., oligo(dT) or hydroxyapatite) that binds to the RNA, e.g., an RNA having a poly(A) sequence, and agitating the contents of the sample container to mix the RNA and the solid affinity material. Liquid is then removed, the process is optionally repeated, and the RNA, e.g., RNA having a poly(A) sequence, is eluted from the solid affinity material. Agitation of the contents of the sample container may involve the introduction of an inert gas into the container, sonication, or the rotation of a disk (e.g., a magnetic disk, e.g., a ferromagnetic disk). These methods provide an RNA, e.g., an RNA having a poly(A) sequence such as an mRNA, having a higher purity and/or a higher yield than an RNA purified using a different method. The invention also features apparatus for purifying a sample including an RNA, e.g., an RNA having a poly(A) sequence, where the apparatus include one or more elements such as a sample container, a reservoir for liquids, a pressure source (e.g., a pump), a valve assembly, a rotatable magnet, and an array for holding collection containers.

### Apparatus for Purifying RNA

The invention features apparatus for purifying an RNA, e.g., having a poly(A) sequence (e.g., an mRNA). including components such as a sample container, liquid reservoir, pressure source, valve assembly, and rotatable magnet. Details of these components are described below.

### Sample Container

The apparatus of the invention employ a sample container, which may be disposable or reusable and cleaned after each use. A sample container may have any useful dimensions and geometry and be made of any useful material (e.g., polypropylene). The sample container may have a height dimension between about 50 mm and about 200 mm (e.g., about 50 mm, 60 mm, 70 mm, 80 mm, 90 mm, 100 mm, 110 mm, 120 mm, 125 mm, 130 mm, 135 mm, 140 mm, 145 mm, 150 mm, 160 mm, 170 mm, 180 mm, 190 mm, or 200 mm). For example, the sample container may measure about 134 mm from top to bottom. The sample container may have a width or radius dimension between about 0.5 mm and about 100 mm (e.g., about 0.5 mm, 1 mm, 1.5 mm, 2 mm, 2.5 mm, 3 mm, 4 mm, 5 mm, 10 mm, 15 mm, 20 mm, 22 mm, 25 mm, 27 mm, 30 mm, 32 mm, 35 mm, 37 mm, 40 mm, 45 mm, 50 mm, 60 mm, 70 mm, 80 mm, 90 mm, and 100 mm). The width or radius of the sample container may vary along its height. For example, the sample container may have a width of about 4 mm at or near its bottom, a width of about 27 mm between the top and bottom, and a width of about 40 mm at or near its top. The width or radius may vary continuously or discontinuously along the height of the sample container. The sample container may have a length dimension between about 0.5 mm and about 100 mm (e.g., about 0.5 mm, 1 mm, 1.5 mm, 2 mm, 2.5 mm, 3 mm, 4 mm, 5 mm, 10 mm, 15 mm, 20 mm, 22 mm, 25 mm, 27 mm, 30 mm, 32 mm, 35 mm, 37 mm, 40 mm, 45 mm, 50 mm, 60 mm, 70 mm, 80 mm, 90 mm, and 100 mm). The length of the sample container may vary along its height. In some embodiments, the sample container has a cross sectional area between about 5 mm² and about 5000 mm² (e.g., about 5 mm², 7 mm², 10 mm², 12 mm², 15 mm², 17 mm², 20 mm², 30 mm², 40 mm², 50 mm², 60 mm², 70 mm², 80 mm², 90 mm², 100 mm², 200 mm², 300 mm², 400 mm², 500 mm², 525 mm², 550 mm², 575 mm², 600 mm², 700 mm², 80 mm², 900 mm², 1000 mm², 1250 mm², 1500 mm², 1750 mm², 2000 mm², 3000 mm², 4000 mm², or 5000 mm²). The cross-sectional area may vary continuously or discontinuously along the height of the sample container. In some embodiments, the sample container has a volume between about 10 cm³ and about 150 cm³ (e.g., about 10 cm³, 20 cm³, 30 cm³, 40 cm³, 50 cm³, 60 cm³, 70 cm³, 80 cm³, 90 cm³, 100 cm³, 110 cm³, 120 cm³, 130 cm³, 140 cm³, or 150 cm³).

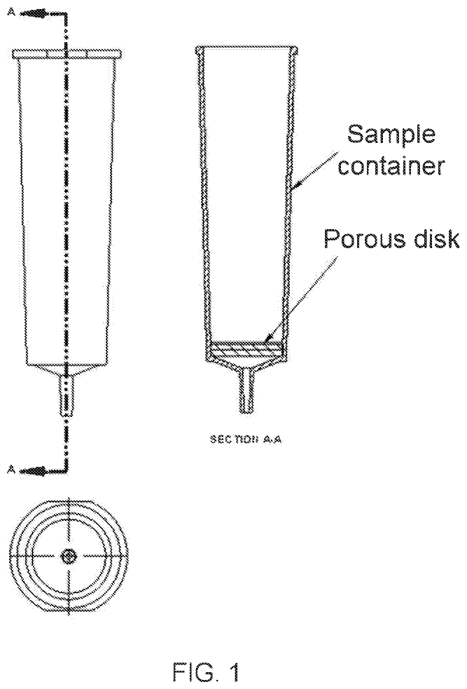

The sample container may include one or more conical portion and/or cylindrical or tubular portions. One or more cylindrical or tubular portions may have the same or different dimensions and may be connected by a conical portion. For example, a cylindrical portion having a first radius may be connected via a conical section to a cylindrical portion having a second radius that is smaller than the first radius. A cylindrical portion may have a diameter between about 0.5 mm and about 100 mm (e.g., about 0.5 mm, 1 mm, 1.5 mm, 2 mm, 2.5 mm, 3 mm, 4 mm, 5 mm, 10 mm, 15 mm, 20 mm, 22 mm, 25 mm, 27 mm, 30 mm, 32 mm, 35 mm, 37 mm, 40 mm, 45 mm, 50 mm, 60 mm, 70 mm, 80 mm, 90 mm, and 100 mm) and/or a height between about 50 mm and about 200 mm (e.g., about 50 mm, 60 mm, 70 mm, 80 mm, 90 mm, 100 mm, 110 mm, 120 mm, 125 mm, 130 mm, 135 mm, 140 mm, 145 mm, 150 mm, 160 mm, 170 mm, 180 mm, 190 mm, or 200 mm). The sample container may also include a tapered cylindrical portion (e.g., a cylindrical portion having a larger radius at one end than at the opposite end), as shown in FIG. 1.

The sample container may include a portion in the form of a regular or oblique prism, such as a rectangular prism. Such a sample container may include one or more fillets or chamfers.

The sample container includes one or more openings. For example, the sample container may include an opening disposed at or near the top of the sample container, and/or an opening disposed at or near the bottom of the sample container. An opening may have any useful size and geometry. For example, an opening may be circular and have a diameter between about 0.5 mm and about 50 mm (e.g., about 0.5 mm, 1 mm, 1.5 mm, 2 mm, 2.5 mm, 3 mm, 4 mm, 5 mm, 10 mm, 15 mm, 20 mm, 22 mm, 25 mm, 27 mm, 30 mm, 32 mm, 35 mm, 37 mm, 40 mm, 45 mm, or 50 mm). Alternatively, an opening may be triangular, square, hexagonal, star shaped, or any other shape. Openings may be open to atmosphere at all times or may be sealed, e.g., with a cap, lid, valve, or cover.

In some embodiments, a sample container is connected to or is configured to engage with one or more caps, lids, covers, windows, tubes, connectors, valves, or other components. For example, the sample container may be connected to or configured to connect to a component at an opening disposed at or near its top or bottom. The sample container may include one or more threaded portions or portions including one or more grooves, notches, depressions, or ridges. Threaded or grooved portions may be disposed on the exterior or interior of the sample container. For example, the sample container may have a threaded or grooved portion near an opening configured to accept a cap or lid with complementary threading or grooves. The sample container may also or alternatively include or be configured to engage with one or more gaskets, o-rings, septa, springs, clasps, or other engagement members. Attachment of a cap, lid, cover, or other component to the sample container may form a seal between the components.

The sample container may include an expanded portion to accommodate, e.g., overfilling or expansion of materials upon addition of liquids or gas to the sample container. The sample container may include a filter or frit to, e.g., retain the solid affinity material. For example, the sample container may include a filter or frit disposed at or near its bottom to prevent solid affinity material from exiting through an opening disposed at or near its bottom, as shown in FIG. 1. The filter or frit may include pores of, for example 10 μm. In other embodiments, the sample container includes one or more support elements such as one or more ridges to support a filter for retaining solid affinity material.

The sample container may include one or more solid affinity materials, e.g., hydroxyapatite or oligo(dT). Additional details of affinity materials are provided herein.

The apparatus for purifying an RNA (e.g., an RNA having a poly(A) sequence, such as an mRNA) may include more than one sample container that have the same or different geometry, dimensions, and other features. For example, an apparatus may include two, three, four, five, six, seven, eight, nine, ten, or more sample containers.

Alternatively, a sample container may be one of a plurality of sample containers within an array of sample containers. Such sample container arrays may be integrally formed, e.g., as a 96-well plate or variant thereof. For example, a sample container may be well of a 96-well filter plate.

### Reservoirs

The apparatus described herein may include one or more reservoirs for housing a liquid. A reservoir may have any useful geometry and dimensions and be formed of any useful materials. In some embodiments, the reservoir is fluidically connected to or configured to be fluidically connected to the sample container via one or more portions of tubing. A valve assembly, gauge, or flow meter may be disposed between the sample container and the reservoir.

The apparatus may include multiple reservoirs for housing different materials. These reservoirs may be of the same of different geometries, dimensions, and/or materials. For example, the apparatus may include a first reservoir for housing a liquid for suspending the solid affinity material (e.g., water, acetonitrile, acetone, or methanol) and a second reservoir for housing a reagent, buffer, or eluting liquid. A reservoir may also be configured to collect and house waste materials.

### Pressure Generating Source

The apparatus described herein may further include or be otherwise coupled to a pressure generating source (e.g., a pump). The pressure generating source may be configured to be in fluid communication one or more other components of the apparatus, such as the sample container, reservoir, or valve assembly. Materials may be separated from the pressure generating source with one or more filters, membranes, and/or other physical elements known in the art. The pressure generating source may also be coupled to a reservoir for collecting waste materials and a component to prevent material from the reservoir from contaminating the pressure generating source.

The pressure generating source may be a low pressure generating source. For example, the pressure generating source may be capable of providing vacuum and/or suction. Pressure generating sources may include one or more peristaltic pumps, syringe pumps, rotary pumps, momentum transfer pumps, diffusion pumps, scroll pumps, and/or diaphragm pumps. The pressure generating source may be a positive displacement or infusion pump such as a peristaltic pump or a syringe pump. In some embodiments, a low pressure generating source may include a house or central vacuum system. In other embodiments, a suction source may include a wall or portable suction device. The pressure generating source may be of any useful size, pumping speed, and geometry and, if included as a component of an apparatus for purifying an RNA, may be disposed in any useful location.

The pressure generating source may be configured to remove waste materials such as liquids from a sample container or reservoir. A filter may prevent waste materials from leaving a waste collection reservoir and possibly aspirating within the pressure generating source (e.g., vacuum pump). The pressure generating source may also or alternatively be configured to pull air or other gas into an apparatus, to transfer a liquid or solution from a reservoir to the sample container, and/or to remove liquid from the sample container. One or more valve assemblies may be included in the apparatus to permit switching between one or more different applications. For example, when the valve assembly is in a first configuration, the pump may be used to transfer a liquid from a reservoir to the sample container, but when the valve assembly is in a second configuration, the pump may be used to remove liquid from the sample container.

The pressure generating source may be configured to provide air or gas to the sample container. The pressure generating source may be fluidically connected to a port in contact with a source of gas (e.g., a pressurized chamber, house gas lines, or gas cylinder). A valve assembly and/or flow meter or other pressure gauge may be used to control the amount of gas introduced into the apparatus. One or more components or regions of the apparatus may be held at positive pressure, such that suction must be applied to draw additional gas into the one or more components or regions of the apparatus. Alternatively, one or more components or regions of the apparatus may be held at negative pressure. Upon suctioning or metering of gas into a region of the apparatus, the pump may facilitate the transfer of the gas to the sample container via, e.g., a fluidic connection to an opening in the sample container (e.g., tubing, such as polyethylene tubing). The addition of gas to a sample container containing a solid affinity material may cause agitation or displacement of the solid affinity material. Gas may be removed from a sample container via an opening in the sample container (e.g., by removing or venting a cap, lid, valve, or cover forming a seal at an opening) or by suction provided by a pressure generating source.

The apparatus for purifying an RNA (e.g., an RNA having a poly(A) sequence, such as an mRNA) may include more than one pressure generating source. For example, an apparatus may include at least two peristaltic pumps, at least two syringe pumps, or at least one peristaltic pump and at least one syringe pump.

### Valve Assembly

The apparatus described herein may include a valve assembly. A valve assembly is a fluid control mechanism used to control the flow of fluids throughout the apparatus. The valve assembly includes a valve to stop, start, or throttle fluid flow. The valve assembly may include any useful valve type, including but not limited to a ball valve, diaphragm valve, and needle valve. In addition to a valve, the valve assembly may include components such as a casing, an electrical or mechanical actuation mechanism (e.g., an electric motor, a hydraulic or pneumatic component, and a solenoid) and associated components (e.g., electric connections and cables), sensors, fasteners (e.g., screws, bolts, clips, and clamps), and mechanical connectors.

A valve assembly may be fluidically connected to one or more components of an apparatus, such as a sample container, a reservoir, an outlet for collection or waste, an inlet for a gas, and a pump. In some embodiments, the apparatus for purifying an RNA includes a sample container, a reservoir, an inlet for air or an inert gas, and an outlet for collection or waste, all of which are fluidically connected to a valve assembly. The valve assembly may be configured to allow, separately, liquid transfer between the sample container and the reservoir, liquid transfer between the sample container and the outlet, and gas transfer between the inlet port and the sample container. A pump (e.g., a peristaltic or syringe pump) may be used to facilitate fluid transfers between components of the apparatus.

The apparatus for purifying an RNA (e.g., an RNA having a poly(A) sequence, such as an mRNA) may include more than one valve assembly. For example, an apparatus may include two, three, four, five, or more valve assemblies.

### Agitators

An apparatus for purifying an RNA may include a mechanical agitator. Mechanical agitators may be useful for physically disrupting the contents of the sample container to cause mixing and include, for example, propellers, paddles, stirrers, and disks. The mechanical agitator may have any useful dimensions and geometry, e.g., to effectively mix a volume within a sample container, and may comprise any useful material. In some embodiments, the mechanical agitator includes a magnet or a magnetizable material. A mechanical agitator such as a magnet or magnetizable component may be in the form of a disk, cylinder, rod, bar, or capsule.

A magnet or magnetizable component may include any useful magnetic or magnetizable material. Many magnetic and magnetizable materials are metals or metal alloys. For example, a magnet may include iron or an alloy of one or more of aluminum, nickel, iron, cobalt, neodymium, boron, and samarium. In some embodiments, a magnet includes Alnico, an alloy of aluminum, nickel, cobalt, and iron.

The mechanical agitator may include a coating comprising, for example, polytetrafluoroethylene (e.g., Teflon), glass, or another chemically inert material.

An apparatus for purifying an RNA and including a mechanical agitator may include one or more components useful for causing the mechanical agitator to move. For example, the apparatus may include a motor, an electromagnetic field generator, and/or an actuation component (e.g., a knob, switch, lever, or dial). A magnet or magnetizable component can be made to rotate in the presence of one or more stationary electromagnets or permanent magnets or a rotating electromagnetic field created by, e.g., a rotating magnet. Thus, the apparatus may include one or more additional magnets and/or other components for generating a rotating electromagnetic field, such as a magnetic resuspension plate (V&P Scientific). In some embodiments, rotation of a magnet or magnetizable component is effected via electrical or mechanical actuation, and the apparatus includes electrical or mechanical actuation components.

### Affinity Materials

An apparatus for purifying an RNA may include or be configured to accept one or more affinity materials. The affinity material may be a solid affinity material.

In some embodiments, the solid affinity material includes oligo(dT). Oligo(dT) is a short sequence of deoxy-thymine nucleotides capable of binding or associating with a poly(A) sequence. Oligo(dT) sequences may include, for example, twelve or more nucleotides. Oligo(dT) sequences may be covalently attached to beads, e.g., polymer beads, optionally via a linker. Methods of attaching oligo(dT) sequences to polymer beads are known in the art. Beads may be substantially spherical and may be of any useful dimension. For example, the beads may have a size of about 100 or fewer μm (e.g., 100 μm, 90 μm, 80 μm, 70 μm, 60 μm, 50 μm, 45 μm, 40 μm, 30 μm, 20 μm, 10 μm, 5 μm, or 1 μm). Beads may be monodisperse or polydisperse. In some instances, oligo(dT) sequences are attached to magnetic particles.

In some embodiments, the solid affinity material includes hydroxyapatite. Hydroxyapatite is a calcium phosphate having the formula [Ca₅(PO₄)₃(OH)]₂. Hydroxyapatite may include particles with a size of about 100 or fewer μm (e.g., 100 μm, 90 μm, 80 μm, 70 μm, 60 μm, 50 μm, 45 μm, 40 μm, 30 μm, 20 μm, 10 μm, 5 μm, or 1 μm). Hydroxyapatite particles may be of any useful shape. For example, hydroxyapatite particles may be substantially spherical or cylindrical or may have hexagonal cross-sections. Hydroxyapatite particles may also be irregularly shaped. Hydroxyapatite particles may be porous, and may have pore diameters of up to 200 nm (e.g., 200 nm, 150 nm, 120 nm, 100 nm, 80 nm, or 50 nm). Without wishing to be bound by theory, the phosphate backbone of an RNA may have affinity for calcium ions of hydroxyapatite while simultaneously being repelled by the phosphate of the hydroxyapatite.

The solid affinity material may have a higher affinity for RNA having a poly(A) sequence (e.g., mRNA) than for other materials, including other RNA (e.g., rRNA and tRNA). For example, the solid affinity material may have an affinity for an RNA having a poly(A) sequence (e.g., mRNA) that is 2, 3, 4, 5, 6, 7, 8, 9, 10, 20, 50, 100, or more fold higher than it is for another component in the sample (e.g., a contaminant, reagent, other RNA, or other component). In some embodiments, a solid affinity material including oligo(dT) may bind RNA at 2-10 mg/ml, e.g., 4-5 mg/ml, of material. In other embodiments, a solid affinity material including hydroxyapatite may bind RNA at 0.1-5 mg/ml, e.g., about 0.8 mg/ml, of material.

### Other Components

An apparatus for purifying an RNA may include components in addition to those described in the preceding sections. For example, the apparatus may include one or more electrical components, cables, tubing, fasteners, connectors, containers for collection of waste or other materials, caps, lids, covers, temperature control (e.g., heating) elements, flow meters, liquid handlers (e.g., a liquid handling robot), computers (e.g., a computer programmed to control the valve assembly and/or pressure source), computer control components, screens, casings, and housings.

In some embodiments, the apparatus includes a temperature control. A temperature control may include a bath (e.g., a water or solvent bath), an electric heater, heating tape, a thermocouple, a sensor, a jacket, insulation, or any other useful element. A temperature control may surround all or a portion of a sample container or reservoir. For example, the sample container may be wrapped with a heating element such as a heating jacket. Alternatively, a surface or portion thereof of a sample container or reservoir may be exposed to a heating element. For instance, a reservoir may be disposed on a heatable plate or other heating element. A sample container or reservoir may also be disposed in a container for housing a solvent (e.g., a solvent bath). Tubing in the apparatus may also be in contact with a heating element such as heating tape or a heating jacket.

In some embodiments, the apparatus includes one or more containers for collection of waste or other materials. These containers may be of any useful shape and dimensions and may be made of any useful materials. In some embodiments, containers for collection of waste or other materials are test tubes or vials. Containers for collection of waste or other materials may be capable of accepting any volume of liquid. For example, collection containers may be capable of accepting a volume of 1 or more ml (e.g., 1 ml, 2 ml, 5 ml, 10 ml, 15 ml, 20 ml, or more). Collection containers include at least one opening through which waste or other materials can be added and may include a cover, lid, valve, or cap to block the opening while materials are not being added. If more than one collection container is present, the collection containers may be organized in an array, and/or a mechanism for transferring the collection containers between different areas of the apparatus may be provided. For example, a mechanical track, carousel, robotic gantry, or other mechanism may be used to position a first collection container such that it can collect materials from, e.g., the sample container. After a volume of materials (e.g., waste) is added to the first collection container, the transfer mechanism can be used to move the first collection container to a different location and to position a second collection container such that it can collect materials from, e.g., the sample container.

In some embodiments, the apparatus includes a computer programmed to control a valve assembly. The computer may utilize software configured to control actuation of the valve to allow liquid or gas transfer between various components of the apparatus at predetermined intervals, or upon user input. The apparatus may also include computer control and monitoring components such as screens, displays, interfaces, and manual manipulators.

### Materials

The apparatus described herein may comprise any useful materials. Such materials may include, e.g., polystyrene, polypropylene, polyvinyl chloride, or combinations thereof. Polymers and/or plastics of the invention may be composite materials in which additives to the polymers and/or plastics, such as ceramics or particles, alter the mechanical properties.

Elements of the invention may also include and/or be formed from glass. For example, an apparatus of the invention may include a reservoir made wholly or partially from glass.

In some embodiments, the liquid level detector, liquid injector, tubing, pressure source, valve assembly, reservoirs, and other components may include and/or be formed from any useful metal or metal alloy, e.g., stainless steel or aluminum.

### Configurations

The apparatus of the invention may include some or all of the components described above.

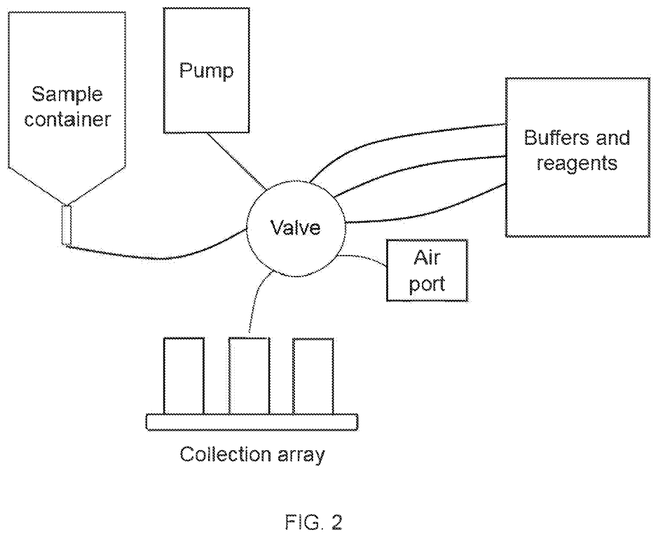

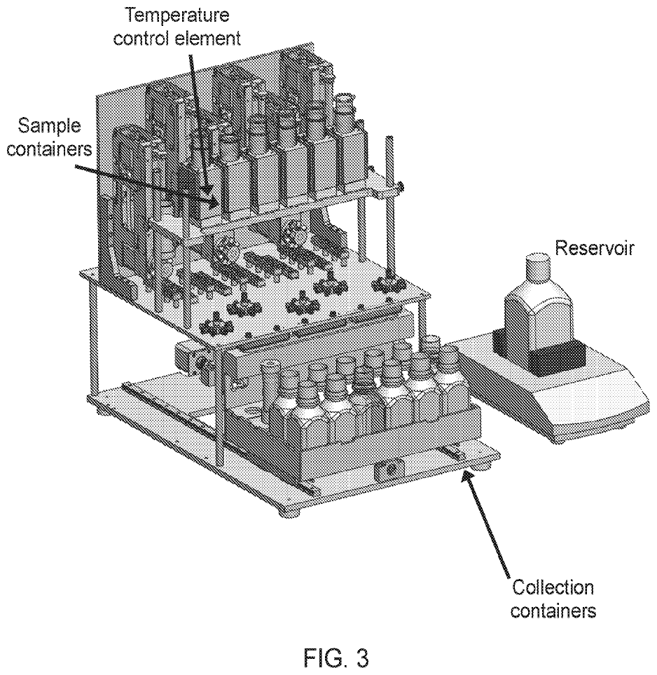

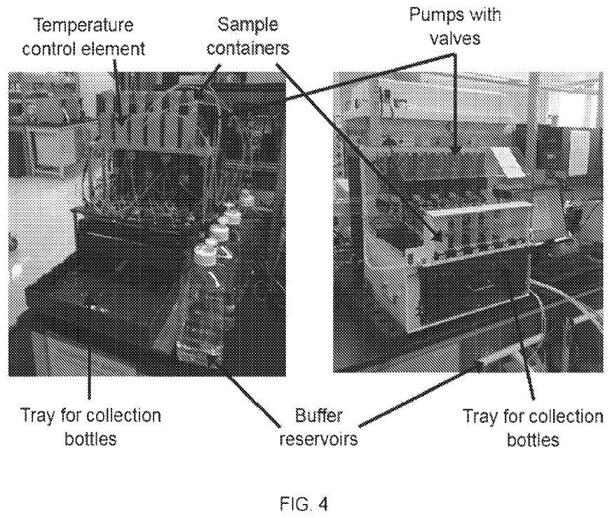

In some embodiments, an apparatus for purifying a sample including an RNA (e.g., an RNA having a poly(A) sequence, such as an mRNA) includes one or more sample containers for depositing the sample, one or more reservoirs for housing a liquid (e.g., a buffer or reagent), a pump (e.g., a peristaltic pump), an outlet for collection or waste, an inlet (e.g., air port) for a gas, and a valve assembly. The sample container may include a first opening disposed at or near its top and a second opening disposed at or near its bottom. The valve assembly may be fluidically connected to the second opening, the reservoir, an outlet for collection or waste, and an inlet for a gas, where the valve assembly is configured to allow, separately, liquid transfer from the sample container and the reservoir, liquid transfer from the sample container and the outlet, and gas transfer from the inlet to the sample container. FIGS. 2-4 illustrate such an apparatus. For example, the apparatus may include one or more additional sample containers, each including a first opening disposed at or near its top and a second opening disposed at or near its bottom. The valve assembly may be fluidically connected to the second opening of the one or more additional sample containers and configured to allow liquid transfer between the one or more additional sample containers and the reservoir or between the one or more additional sample containers and the outlet or gas transfer between the inlet and the one or more additional sample containers. The apparatus may also comprise one or more heaters or temperature control elements for the one or more sample containers. In some embodiments, the sample container includes or is configured to accept a solid affinity material, e.g., including hydroxyapatite or oligo(dT), as described herein.

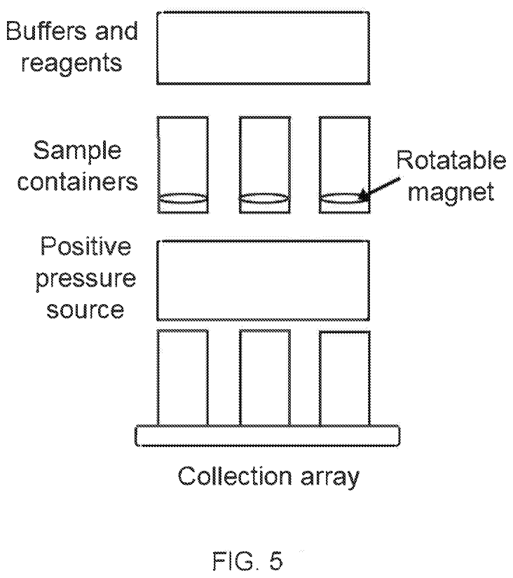

In some embodiments, an apparatus for purifying a sample including an RNA (e.g., an RNA having a poly(A) sequence, such as an mRNA) includes a sample container for depositing the sample, a mechanical agitator (e.g., a disk or other shaped object as described herein) disposed in the sample container, and a positive pressure source disposed below the sample container. FIG. 5 is an illustration of such an apparatus. The apparatus also include an element, e.g., a rotatable magnet, to actuate mechanical agitation. The sample container may include a first opening disposed at or near its top and a second opening disposed at or near its bottom and may include or be configured to accept a solid affinity material, e.g., oligo(dT), as described herein. The positive pressure source may be configured to prevent the contents of the sample container from exiting the sample container and/or to eject components through an opening at or near the top of the sample container. The apparatus may include an array of sample containers (e.g., a filter or microarray plate). In some embodiments, the apparatus includes a reservoir for a liquid that is in fluid communication with an opening of the sample container (e.g., the first opening). The apparatus may include, or be configured for use with, an instrument for automatically handling one or more liquids. For example, the apparatus may include, or be configured for use with, a liquid handling robot (e.g., a Hamilton robot). A liquid handling robot may be useful for transferring sample and/or other liquids (e.g., buffers and reagents) from a first container (e.g., a reservoir) to a sample container or an array of sample containers (e.g., a filter or microarray plate). The apparatus may also include an array for holding collection containers for collecting material from an opening of the sample container (e.g., the second opening). In some embodiments, the apparatus may include, or be configured for use with, an additional pressure generating source. The additional pressure generating source may be disposed above the sample container and may be used to add liquid to or remove liquid from a sample container.

### Methods for Purifying RNA

The invention features methods for purifying an RNA (e.g., an RNA having a poly(A) sequence, such as an mRNA, as described herein) including, for example, adding a liquid to a sample container including the sample and a solid affinity material (e.g., oligo(dT) or hydroxyapatite) that binds to the RNA, agitating the contents of the sample container to mix the RNA and the solid affinity material, removing the liquid from the sample container, optionally repeating the process, and eluting the RNA from the solid affinity material.

In some embodiments, the sample container is provided with a solid affinity material. Alternatively, the solid affinity material may be added to the sample container via an opening disposed at or near the top of the sample container. The solid affinity material may be packed into the sample container. In some instances, the solid affinity material may be packed onto a filter or frit disposed inside the sample container, e.g., to prevent the solid affinity material from exiting the sample container via an opening disposed at or near the bottom of the sample container.

The sample including an RNA (e.g., an RNA having a poly(A) sequence, such as an mRNA) may be added to the sample container via an opening disposed at or near the top of the sample container. The sample may be loaded onto the top of a packed bed of solid affinity material.

Liquid (e.g., a reagent or buffer) may be added to the sample container from a reservoir. Liquid may be introduced to the sample container from an opening disposed at or near the top or the bottom of the sample container, e.g., using an automated liquid handler or a pump and valve assembly. A liquid added to the sample container may have a relatively high salt content. For example, a solution added to the chamber may include 10 mM Tris, 1 mM EDTA, and 0.5 M NaCl and have a pH of 7.4. A liquid with a lower salt content may be added to the sample container before or after addition of the higher salt content liquid. For example, a solution including 10 mM Tris, 1 mM EDTA, and 0.1 M NaCl and having a pH of 7.4 may be added to the sample container after addition and subsequent removal of the 0.5 M NaCl solution. Alternatively or in addition, water may be added to the sample container. For example, warm water may be used to elute an RNA of interest (e.g., RNA having a poly(A) sequence, such as an RNA) from the sample container.

The contents of the sample container (e.g., sample, solid affinity material, and liquid) are agitated for mixing. Agitation may cause a higher amount of the RNA of interest (e.g., RNA having a poly(A) sequence, such as an mRNA) to bind to the solid affinity material, resulting in enhanced purity and yield of the RNA of interest. Without wishing to be bound by theory, agitation of the contents of the sample container may expose a higher surface area of solid affinity material to the sample, thus providing additional opportunities for binding of the RNA of interest. In some embodiments, agitation may involve introducing a gas (e.g., air, nitrogen, carbon dioxide, helium, argon, or neon) into the sample container. In other embodiments, agitation may involve moving the sample container. For example, the sample container may be shaken, rotated, oscillated, centrifuged, or otherwise moved to cause movement of its contents. Movement of the sample container may be achieved via automated or manual action. Agitation may also involve sonication. For example, the sample container may be placed within a bath and sonic frequencies applied to effect movement of the contents of the sample container. In other embodiments, the contents of the sample container may be agitated by movement of a component included within the sample container (e.g., a mechanical agitator). For example, the sample container may include a propeller, paddle, stirrer, disk, or other mechanical agitator that physically disrupts the contents of the sample container and causes mixing. Movement of the mechanical agitator may be brought about through use of a motor or application of an electromagnetic field (e.g., a mechanically or electrically generated field, such as a rotating electromagnetic field produced by a magnetic resuspension plate). The mechanical agitator may be a magnetic or magnetizable disk (e.g., as described herein) that moves inside the sample container in response to a rotating electromagnetic field. A mechanical agitator such as a rotating magnet may move in an ordered or random fashion and may translate vertically within the sample container (e.g., from a position at or near the bottom of the sample container to a position at or near the top of the sample container) or may remain in a fixed vertical position during agitation. The contents of the sample container may be agitated at any useful intensity for any useful amount of time (e.g., seconds, minutes, hours, or days). Agitation may result in resuspension of the contents of sample container, e.g., the disruption of a packed bed of solid affinity material.

After agitation, liquid may be removed from the sample container by evacuation, evaporation, draining (elution), or any other useful method. For example, an opening disposed at or near the bottom of a sample container may be closed (e.g., with a lid, cover, valve, or cap) during the addition and agitation steps and subsequently opened or vented (e.g., by removal of the lid, cover, or cap by, for example, opening a valve, flipping a switch, or another automated or manual motion) to permit liquid to exit the bottom of the sample container. A pump and valve assembly may be used to control removal of liquid from the sample container. In an apparatus in which contents are retained within the sample container by means of a positive pressure source, liquid can be removed by removing or reducing the positive pressure or by increasing the positive pressure to cause liquid to exit the opposite end of the sample container. Solid affinity material, to which the RNA of interest (e.g., RNA having a poly(A) sequence, such as an mRNA) should be bound, may be blocked from exiting the sample container by a frit or filter (e.g., a porous disk) disposed inside the sample container. The solid affinity material remaining in the sample container may form a packed bed after removal of the liquid. The sample container may be substantially free of liquid after draining of liquid through an opening disposed at or near the bottom of the container. In other words, the contents remaining in the sample container may be substantially dry.

In some embodiments, upon draining liquid from an opening in the sample container, the opening is blocked (e.g., with a lid, cover, valve, or cap) and the process is repeated. Additional liquid may be added to the sample container and the sample container contents once again agitated prior to removal of the liquid. The additional liquid may be the same or different as the liquid used in the first liquid addition step. In some embodiments, the additional liquid may have a different salt content, pH, and/or polarity than the liquid used in the first liquid addition step. Subsequent agitation steps may be of equal, greater, or lesser intensity and/or duration as the first agitation step and may involve the same or different agitation method.

Following one or more cycles of liquid addition, agitation, and liquid removal, an eluting liquid capable of disrupting the binding between the RNA having a poly(A) sequence (e.g., mRNA) and the solid affinity material is added to elute the RNA having a poly(A) sequence from the solid affinity material. The eluting liquid may have, for example, a low salt content. Without wishing to be bound by theory, a low salt buffer may destabilize the binding interaction between an RNA having a poly(A) sequence and a solid affinity material. In some embodiments, the eluting liquid includes Tris-HCl, while in others the eluting liquid is water. As with other liquids added to the sample container, the eluting liquid may be heated or cooled to any useful temperature using, e.g., a temperature control element described herein.

### Samples

The present invention is directed to methods and apparatus for purifying a ribonucleic acid (RNA) from a sample. The RNA may include a poly(A) sequence, which is a sequence including only adenine nucleobases or modified versions thereof (e.g., a poly(A) tail, region, or signal). The poly(A) sequence is often disposed at the 3′ end of an RNA and is often a component of messenger RNA (mRNA). Without wishing to be bound by theory, the poly(A) sequence of mRNA protects the mRNA from degradation and facilitates nuclear export, translation, and termination of transcription. Details of poly(A) sequences are provided herein.

In addition to an RNA (e.g., an RNA having a poly(A) sequence, such as an mRNA), samples purified using the methods and apparatus described herein may include, for example, a surfactant (e.g., sodium dodecyl sulfate), a buffer (e.g., a sodium acetate buffer), a chelating agent (e.g., EDTA), a solvent (e.g., chloroform, ethanol, and phenol), other types of RNA (e.g., ribosomal RNA (rRNA) and transfer RNA (tRNA)), adenosine triphosphate (ATP), an enzyme (e.g., E. coli Poly(A) Polymerase), or any other component. For example, the sample may include components relevant to in vitro transcription reactions or polyadenylation reactions. The sample may also include DNA or other types of RNA (e.g., rRNA and tRNA) such that purification of the sample using the methods and apparatus described herein isolates RNA having a poly(A) sequence from DNA and total RNA.

### Purity

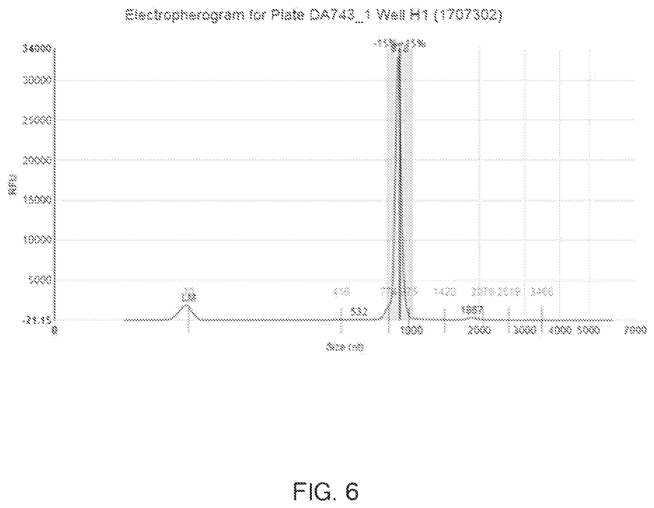

In some embodiments, the apparatus and methods of the present invention may provide an RNA (e.g., an RNA having a poly(A) sequence, such as an mRNA) with a purity of 80% or greater (e.g., 80%, 85%, 90%, 91%, 92%, 93%, 94%, 95%, 96%, 97%, 98%, 99%, 99.5%, or 100%). Purity can be measured using, e.g., an AATI Fragment Analyzer using capillary electrophoresis and quantified as the % area under the curve ±15% around the main peak. FIG. 6 shows an electropherogram of an RNA purified using the methods of the invention. The RNA has a purity of greater than 85%.

### Yield

In some embodiments, the apparatus and methods of the present invention may provide an RNA (e.g., an RNA having a poly(A) sequence, such as an mRNA) with in a yield of 70% or greater (e.g., 70%, 71%, 72%, 73%, 74%, 75%, 76%, 77%, 78%, 79%, 80%, 85%, 90%, 91%, 92%, 93%, 94%, 95%, 96%, 97%, 98%, 99%, 99.5%, or 100%).

### RNA

An RNA of interest (e.g., an RNA having a poly(A) sequence, such as an mRNA) may be naturally or non-naturally occurring and may include one or more alterations. Herein, in a nucleotide, nucleoside, or polynucleotide (such as the polynucleotides of the invention, e.g., mRNA molecules), the terms “alteration” or, as appropriate, “alternative,” refer to alteration with respect to A, G, U or C ribonucleotides. Generally, herein, these terms are not intended to refer to the ribonucleotide alterations in naturally occurring 5′-terminal mRNA cap moieties.

The alterations may be various distinct alterations. In some embodiments, where the polynucleotide is an mRNA, the coding region, the flanking regions and/or the terminal regions (e.g., a 3′-stabilizing region) may contain one, two, or more (optionally different) nucleoside or nucleotide alterations. In some embodiments, an alternative polynucleotide introduced to a cell may exhibit reduced degradation in the cell, as compared to an unaltered polynucleotide.

The polynucleotides of the invention can include any useful alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations may be alterations of ribonucleic acids (RNAs) to deoxyribonucleic acids (DNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

As described herein, in some embodiments, the polynucleotides of the invention do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc.), and/or 3) termination or reduction in protein translation.

The polynucleotides can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors). In some embodiments, the polynucleotides may include one or more messenger RNAs (mRNAs) having one or more alternative nucleoside or nucleotides (i.e., alternative mRNA molecules). Details of these polynucleotides follow.

Nucleobase Alternatives

The alternative nucleosides and nucleotides can include an alternative nucleobase. Examples of nucleobases found in RNA include, but are not limited to, adenine, guanine, cytosine, and uracil. Examples of nucleobases found in DNA include, but are not limited to, adenine, guanine, cytosine, and thymine. These nucleobases can be altered or wholly replaced to provide polynucleotide molecules having enhanced properties, e.g., increased stability such as resistance to nucleases.

Alternative nucleotide base pairing encompasses not only the standard adenine-thymine, adenine-uracil, or guanine-cytosine base pairs, but also base pairs formed between nucleotides and/or alternative nucleotides including non-standard or alternative bases, wherein the arrangement of hydrogen bond donors and hydrogen bond acceptors permits hydrogen bonding between a non-standard base and a standard base or between two complementary non-standard base structures. One example of such non-standard base pairing is the base pairing between the alternative nucleotide inosine and adenine, cytosine, or uracil.

In some embodiments, the nucleobase is an alternative uracil. Exemplary nucleobases and nucleosides having an alternative uracil include pseudouridine (Ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (τm⁵U), 1-taurinomethyl-pseudouridine, 5-taurinomethyl-2-thio-uracil(τm⁵s²Ψ), 1-taurinomethyl-4-thio-pseudouridine, 5-methyl-uracil (m⁵U, i.e., having the nucleobase deoxythymine), 1-methyl-pseudouridine (m¹Ψ), 5-methyl-2-thio-uracil (m⁵s²U), 1-methyl-4-thio-pseudouridine (m¹s⁴Ψ), 4-thio-1-methyl-pseudouridine, 3-methyl-pseudouridine (m³Ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ Ψ), 5-(isopentenylaminomethyl)uracil (inm⁵U), 5-(isopentenylaminomethyl)-2-thio-uracil (inm⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQ0), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

Alterations on the Sugar

The alternative nucleosides and nucleotides, which may be incorporated into a polynucleotide of the invention (e.g., RNA or mRNA, as described herein), can be altered on the sugar of the nucleoside or nucleotide. In some embodiments, the alternative nucleosides or nucleotides include the structure:

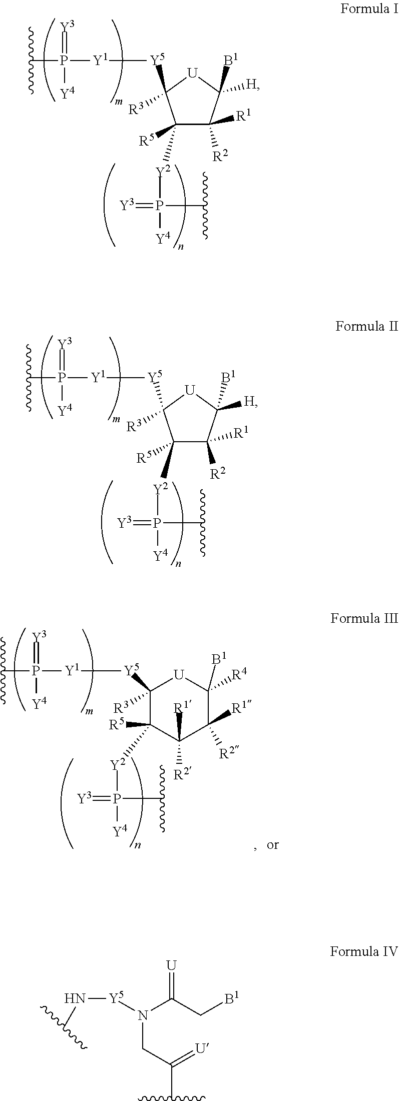

wherein B¹ is a nucleobase;

each U and U′ is, independently, O, S, N(R^{U})ₙᵤ, or C(R^{U})ₙᵤ, wherein nu is 1 or 2 (e.g., 1 for N(R^{U})ₙᵤ and 2 for C(R^{U})ₙᵤ) and each R^{U} is, independently, H, halo, or optionally substituted C₁-C₆ alkyl;

each of R¹, R^{1′}, R^{1″}, R², R^{2′}, R^{2″}, R³, R⁴, and R⁵ is, independently, H, halo, hydroxy, thiol, optionally substituted C₁-C₆ alkyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted amino, azido, optionally substituted C₆-C₁₀ aryl; or R³ and/or R⁵ can join together with one of R¹, R^{1′}, R^{1″}, R², R^{2′}, or R^{2″} to form together with the carbons to which they are attached an optionally substituted C₃-C₁₀ carbocycle or an optionally substituted C₃-C₉ heterocyclyl; each of m and n is independently, 0, 1, 2, 3, 4, or 5;

each of Y¹, Y², and Y³, is, independently, O, S, Se, —NR^{N1}—, optionally substituted C₁-C₆ alkylene, or optionally substituted C₁-C₆ heteroalkylene, wherein R^{N1} is H, optionally substituted C₁-C₆ alkyl, optionally substituted C₂-C₆ alkenyl, optionally substituted C₂-C₆ alkynyl, or optionally substituted C₆-C₁₀ aryl; and

each Y⁴ is, independently, H, hydroxy, protected hydroxy, halo, thiol, boranyl, optionally substituted C₁-C₆ alkyl, optionally substituted 02-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, or optionally substituted amino; and

Y⁵ is O, S, Se, optionally substituted C₁-C₆ alkylene, or optionally substituted C₁-C₆ heteroalkylene;

or a salt thereof.

In some embodiments, the 2′-hydroxy group (OH) can be modified or replaced with a number of different substituents. Exemplary substitutions at the 2′-position include, but are not limited to, H, azido, halo (e.g., fluoro), optionally substituted C₁₋₆ alkyl (e.g., methyl); optionally substituted C₁₋₆ alkoxy (e.g., methoxy or ethoxy); optionally substituted C₆₋₁₀ aryloxy; optionally substituted C₃₋₈ cycloalkyl; optionally substituted C₆₋₁₀ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxy is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein.

Generally, RNA includes the sugar group ribose, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a polynucleotide molecule can include nucleotides containing, e.g., arabinose or L-ribose, as the sugar.

In some embodiments, the polynucleotide of the invention includes at least one nucleoside wherein the sugar is L-ribose, 2′-O-methyl-ribose, 2′-fluoro-ribose, arabinose, hexitol, an LNA, or a PNA.

Alterations on the Internucleoside Linkage

The alternative nucleotides, which may be incorporated into a polynucleotide of the invention, can be altered on the internucleoside linkage (e.g., phosphate backbone). Herein, in the context of the polynucleotide backbone, the phrases “phosphate” and “phosphodiester” are used interchangeably. Backbone phosphate groups can be altered by replacing one or more of the oxygen atoms with a different substituent.

The alternative nucleotides can include the wholesale replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy.

The replacement of one or more of the oxygen atoms at the a position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleoside linkages that may be employed according to the present invention, including internucleoside linkages which do not contain a phosphorous atom, are described herein.

### Poly-A Regions

A poly(A) sequence of an RNA may be comprised entirely or mostly of adenine nucleotides or analogs or derivatives thereof. A polyA sequence may be a tail located adjacent to a 3′ untranslated region of a nucleic acid and need not be at the 3′ terminus.

During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that can be between, for example, approximately 80 to approximately 250 residues long (e.g., 80, 90, 100, 110, 120, 130, 140, 150, 160, 170, 180, 190, 200, 210, 220, 230, 240, or 250 residues long). Poly(A) sequences can also be added after a construct is exported from a cell nucleus. A poly(A) sequence may confer stabilization to the RNA.

Unique poly(A) region lengths may provide certain advantages to the alternative polynucleotides of the present invention. Generally, a poly(A) sequence is greater than 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1100, 1200, 1300, 1400, 1500, 1600, 1700, 1800, 1900, 2000, 2500, and 3000 nucleotides in length). In some cases, the poly(A) sequence of an RNA molecule is 10, 20, 30, 40, 50, 60, 70, 80, 90 or 100% greater in length than the RNA or another feature thereof. The poly(A) sequence may be, for example, 10, 20, 30, 40, 50, 60, 70, 80, or 90% or more of the total length of the RNA or the total length of the RNA minus the poly-A region. Further, engineered binding sites and conjugation of polynucleotides (e.g., RNA molecules) via the Poly(A) binding protein can enhance expression.

Additionally, multiple distinct polynucleotides (e.g., RNA molecules) can be linked together via the PABP (Poly(A) binding protein) through the 3′-end using modified nucleotides at the 3′-terminus of the poly(A) sequence. Transfection experiments can be conducted in relevant cell lines at and protein production can be assayed by ELISA at 12 hours, 24 hours, 48 hours, 72 hours, and 7 days post-transfection.

An RNA (e.g., an RNA having a poly(A) sequence, such as an mRNA) may include a polyA-G Quartet region. The G-quartet is a cyclic hydrogen bonded array of four guanine nucleotides that can be formed by G-rich sequences in both DNA and RNA. The G-quartet may be incorporated at the end of the poly(A) sequence.

### Compositions Including RNA

In some embodiments, an RNA having a poly(A) sequence (e.g., an mRNA) purified using the methods and apparatus of the invention is used in pharmaceutical compositions. For example, an RNA may be encapsulated in a lipid nanoparticle, lipoplex, liposome, polymeric particle, or other composition for administration to a mammal to, e.g., produce a polypeptide of interest in a mammalian cell, tissue, or organ and/or to treat a disease or disorder. Compositions including an RNA having a poly(A) sequence (e.g., an mRNA) purified using the methods and apparatus of the invention may include, for example, one or more cationic and/or ionizable lipids, phospholipids, structural lipids, surfactants, emulsifiers, excipients, PEGylated lipids (e.g., lipids including a polyethylene glycol component), or other components.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all, of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

### Example 1: Purification of an RNA Sample Having a Poly(A) Sequence Involving Air Agitation

A sample container such as that in FIG. 1 is provided. The sample container may be made primarily of polypropylene and include openings at both its top and bottom. The opening at the top of the sample container is approximately 40 mm in diameter. The top cylindrical portion of the sample container is approximately 110 mm high and is tapered such that the diameter decreases to approximately 27 mm at the bottom of the tapered cylinder, where a porous disk is positioned. A conical portion connects the upper tapered cylindrical portion to a lower tapered cylindrical portion having a 2 mm opening at its bottom. The porous disk prevents solid materials from exiting the bottom of the sample container. In some cases, a filter resting on, for example, ridges disposed on the interior of the sample container can be used in place of a porous disk. The openings at the top and bottom of the sample container may be sealed via a cap, lid, valve, or other cover. Tubing (e.g., rubber or plastic tubing) may be attached to either end of the sample container to establish fluid communication with a pump and valve assembly.

A solid affinity material (oligo(dT) or hydroxyapatite) is added to the sample container through the opening at its top. The solid affinity material may be packed onto the porous disk.

A sample including an mRNA of interest is prepared in an IVT or CAP reaction. The sample is added to the sample container through the opening at its top.

A solution is subsequently added to the bottom of the sample container from a reservoir via a pump and valve assembly. The solution may be, for example, a “high-salt” solution including 10 mM Tris, 1 mM EDTA, and 0.5 M NaCl. The contents of the sample container are then agitated by pumping air into the bottom of the sample container. The air may be pumped into the sample container using the same syringe that pumps in buffers and reagents. The pump may move a full syringe stroke (50 ml) through the sample container at a rate of 7.5 ml/second. The agitation resuspends the solid affinity material and exposes the mRNA of interest to a greater surface area of solid affinity material to enhance the binding of the mRNA to the solid affinity material. The pump is subsequently used to remove the liquid from the sample container. A second solution is then added to the bottom of the sample container. The solution may be, for example, a “low-salt” solution including 10 mM Tris, 1 mM EDTA, and 0.1 M NaCl. The agitation and liquid removal steps are then repeated. Liquid addition, agitation, and liquid removal may be further repeated as needed to purify the mRNA.

An eluting liquid (e.g., warm water) is added to the bottom of the sample container from a reservoir via a pump and valve assembly. The eluting liquid causes the mRNA to disassociate from the solid affinity material. Without wishing to be bound by theory, water above 55° C. causes the double stranded junction between the polyA tail of the mRNA and the polyT tail of the solid affinity material (e.g., resin) to separate. The liquid and mRNA can then be collected in a collection container. The purity and yield of the mRNA can then be assessed.

### Example 2: Purification of an RNA Sample Having a Poly(A) Sequence Involving a Mechanical Agitator

A sample container including a mechanical agitator is provided. The sample container may be substantially cylindrical and may be made primarily of polypropylene. The sample container includes openings at both its top and bottom. The openings at the top and bottom of the sample container may be sealed via a cap, lid, valve, or other cover. Tubing (e.g., rubber or plastic tubing) may be attached to either end of the sample container to establish fluid communication with a pump, valve assembly, and/or a reservoir. The sample container is positioned above a positive pressure source. A solid affinity material (oligo(dT)) is added to the sample container through the opening at its top. The positive pressure source prevents the solid affinity material from exiting through the bottom of the sample container.

A sample including an mRNA of interest is prepared for purification. The sample is added to the sample container through the opening at its top.

A solution is subsequently added to the top of the sample container from a reservoir disposed above the sample container. A liquid handling robot can be used to transfer the solution in semi- or fully-automated fashion from the reservoir to the sample container. The solution may be, for example, a “high-salt” solution including 10 mM Tris, 1 mM EDTA, and 0.5 M NaCl. The contents of the sample container are then agitated by causing the mechanical agitator to move. The mechanical agitator may be a magnet or magnetizable disk capable of rotating in the presence of a rotating electromagnetic field. The agitation resuspends the solid affinity material and exposes the mRNA of interest to a greater surface area of solid affinity material to enhance the binding of the mRNA to the solid affinity material. The solution can subsequently be removed by increasing the positive pressure to blow the solution through the opening at the top of the sample container. A second solution is then added to the top of the sample container. The solution may be, for example, a “low-salt” solution including 10 mM Tris, 1 mM EDTA, and 0.1 M NaCl. The agitation and liquid removal steps are then repeated. Liquid addition, agitation, and liquid removal may be further repeated as needed to purify the mRNA.

An eluting liquid (e.g., warm water) is added to the top of the sample container from a reservoir. The eluting liquid causes the mRNA to disassociate from the solid affinity material. The liquid and mRNA can then be collected in a collection container. The purity and yield of the mRNA can then be assessed.

### Example 3: Large-Scale Purification of an mRNA Sample

A sample including 40 mg of an mRNA of interest is prepared via an enzymatic reaction in a 26.7 ml solution. After quenching the reaction, the total volume is 32.27 ml. The sample is then equilibrated to a 500 mM NaCl solution. The sample is divided into two portions, each including 20 mg mRNA, for use in two different reaction conditions. Each portion is combined with 4 ml of dry oligo(dT) resin in a 50% slurry with a 500 mM NaCl buffer (8 ml slurry volume).

The two samples, Sample A and Sample B, are subsequently subjected to different binding conditions: “off-instrument” and “on-instrument” binding. Sample A is subjected to “on-instrument” binding, which involves 30 minutes of bubbling (e.g., agitation by addition of air, as described in Example 1) followed by 20 minutes of rest. Sample B is subjected to “off-instrument” binding, which involves 30 minutes of small radius shaking at 42° C. followed by 20 minutes on an orbital shaker. While the binding of Sample A is carried out within a sample container in an apparatus such as that of Example 1 (e.g., a sample container in fluid communication with the pump, valve assembly, one or more reservoirs, and an air inlet port), the binding of Sample B is carried out in a separate container external to the apparatus. Following binding, Sample B is added to a sample container in an apparatus such as that of Example 1.

Purification proceeds identically for both conditions, as shown in Table 1:

TABLE 1
Large-scale purification of mRNA samples.

| | Elution 1 | | | Elution 2 | | | | |
|---|---|---|---|---|---|---|---|---|
| | | | mass | | | mass | Total mass | |
| | Conc. | Volume | mRNA | Conc. | Volume | mRNA | mRNA | % |
| Condition | (ng/uL) | (mL) | (mg) | (ng/uL) | (mL) | (mg) | Eluted (mg) | Yield |
| Sample A | 635.5 | 24 | 15.3 | 139.4 | 12 | 1.7 | 16.9 | 84.6 |
| Sample B | 654.2 | 24 | 15.7 | 158.2 | 12 | 1.9 | 17.6 | 88.0 |

As shown in Table 1, the yield of mRNA was nearly 85% for Sample A and 88% for Sample B. The slight difference between yields for the two methods may be attributable to the construct dependence for the binding to the solid affinity material.

### Example 4: Automated Small-Scale mRNA Purification

A liquid handler gripper places a 96-well filter plate in a nest on top of a rotating magnet. The 96-well filter plate is pre-filled with ferromagnetic discs (one per well) and 200 μl resin per well. An automated liquid handler pipette dispenses binding buffer to each of the 96 wells. The spinning discs agitate the solution, keeping the resin suspended and unable to sediment to the bottom of the wells. Agitation occurs for 10 minutes, while the mRNA binds to the resin. Upon completion of the binding step, the plate is moved to a positive pressure manifold, which displaces the liquid from the 96-well filter plate into a collection or waste plate.

The liquid handler pipette dispenses a high salt wash buffer into each of the wells, and the agitation process is repeated to provide a first wash to the bound mRNA. After the first wash, the plate is again moved to a positive pressure manifold, where the wash buffer is removed.

The wash step is repeated with a low salt buffer. After removal of the low salt buffer, the agitation is repeated with an elution buffer to elute the purified mRNA from the resin.

### Example 5: Assessment of mRNA Yield Resulting from a dT Process

A sample containing 278.0 μl of clean mRNA was dispensed in each well a 96-well filter plate and an automated dT process according to the invention was carried out. Results from nine wells are shown below in Table 2.

TABLE 2
Recovery and yield of mRNA from automated dT process

| | |
|---|---|
| Average | 197.6 |
| Standard Deviation | 15.4 |
| % CV | 7.8 |
| Recovery (%) | 71.1 |

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

What is claimed is:

1. A method of purifying a sample including an RNA optionally having a poly(A) sequence, the method comprising:
a) adding a liquid to a sample container comprising the sample and a solid affinity material that binds to the RNA;
b) agitating the contents of the sample container to mix the RNA and the solid affinity material;
c) removing the liquid from the sample container; and
d) eluting the RNA from the solid affinity material.

2. The method of claim 1, wherein the RNA has a poly(A) sequence.

3. The method of claim 1 or 2, wherein the RNA is an mRNA.

4. The method of claim 3, wherein the mRNA includes one or more alternative nucleosides or nucleotides.

5. The method of any one of claims 1-4, wherein the RNA includes one or more of a 5′ cap structure, a Kozak sequence, a 3′ UTR, and a 5′ UTR.

6. The method of any one of claims 1-5, wherein the solid affinity material comprises oligo(dT).

7. The method of claim 6, wherein the particles comprising oligo(dT) have a size of between about 1 μm and 100 μm.

8. The method of claim 7, wherein the particles comprising oligo(dT) have a size of about 45 μm.

9. The method of any one of claims 1-5, wherein the solid affinity material comprises hydroxyapatite.

10. The method of claim 9, wherein the hydroxyapatite particles have a size of between about 1 μm and 100 μm.

11. The method of claim 10, wherein the particles comprising hydroxyapatite have a size of about 40 μm.

12. The method of any one of claims 1-11, wherein the sample container includes a substantially cylindrical portion.

13. The method of any one of claims 1-12, wherein the sample container includes a conical portion.

14. The method of any one of claims 1-13, wherein the sample container comprises an opening disposed at or near its top and an opening disposed at or near its bottom.

15. The method of any one of claims 1-14, wherein the sample container comprises a porous filter or frit disposed at or near its bottom.

16. The method of any one of claims 1-15, wherein, prior to step (a), the sample is loaded onto the top of a packed bed of the solid affinity material.

17. The method of any one of claims 1-16, wherein step (a) comprises introducing the liquid into the sample container from the bottom of the container.

18. The method of any one of claims 1-17, wherein step (b) comprises introducing an inert gas into the container.

19. The method of claim 18, wherein the inert gas is air, nitrogen, argon, or carbon dioxide.

20. The method of any one of claims 1-17, wherein step (b) comprises sonication.

21. The method of any one of claims 1-7 and 12-15, wherein the sample container comprises a mechanical agitator, and step (b) comprises moving the mechanical agitator.

22. The method of claim 21, wherein the mechanical agitator is a magnetic or magnetizable disk, and step (b) comprises rotating the disk.

23. The method of any one of claims 1-22, wherein the sample container is disposed above a positive pressure source to prevent leaking of the liquid.

24. The method of any one of claims 1-23, wherein, after step (c), the container is substantially free of the liquid.

25. The method of any one of claims 1-24, wherein steps (a)-(c) are repeated at least once prior to step (d).

26. The method of claim 16, wherein steps (a) and (b), in combination, disrupt the packed bed and mix the solid affinity material with the liquid, and step (c) results in reformation of the packed bed.

27. An apparatus for purifying a sample including an RNA, the apparatus comprising:
a) a sample container for depositing the sample, wherein the sample container comprises a first opening disposed at or near its top and a second opening disposed at or near its bottom;
b) a reservoir for housing a liquid; and
c) a valve assembly, wherein the valve assembly is fluidically connected to the second opening, the reservoir, an outlet for collection or waste, and an inlet for an inert gas, wherein the valve assembly is configured to allow, separately, liquid transfer from the sample container and the reservoir, liquid transfer from the sample container and the outlet, and gas transfer from the inlet to the sample container.

28. The apparatus of claim 27, further comprising a pump.

29. The apparatus of claim 28, wherein the pump is a peristaltic pump or a syringe pump.

30. The apparatus of any one of claims 27-29, further comprising a computer programmed to control the valve assembly.

31. The apparatus of any one of claims 27-30, further comprising one or more additional sample containers, each comprising a first opening disposed at or near its top and a second opening disposed at or near its bottom, wherein the valve assembly is fluidically connected to the second opening, the reservoir, an outlet for collection or waste, and an inlet for an inert gas, wherein the valve assembly is configured to allow, separately, liquid transfer from the sample container and the reservoir, liquid transfer from the sample container and the outlet, and gas transfer from the inlet to the sample container.

32. The apparatus of any one of claims 27-31, further comprising a heater for the sample container.

33. An apparatus for purifying a sample including an RNA, the apparatus comprising:
a) a sample container for depositing the sample, wherein the sample container comprises a first opening disposed at or near its top and a second opening disposed at or near its bottom;
b) a mechanical agitator disposed in the sample container; and
c) a positive pressure source disposed below the sample container.

34. The apparatus of any one of claim 33, wherein the mechanical agitator is a magnetic or magnetizable disk.

35. The apparatus of any one of claims 33-34, further comprising a reservoir for a liquid in fluid communication with the first opening.

36. The apparatus of any one of claims 33-35, further comprising an array for holding collection containers to capture eluate from the second opening.

37. The apparatus of any one of claims 33-36, further comprising a pressure source configured to cause liquid in the sample container to exit the second opening.

38. An RNA purified using the method of any one of claims 1-26.
